(19) United States
(12) Patent Application Publication
(10) Pub. No.: US 2013/0317523 A1
(43) Pub. Date: Nov. 28, 2013

(54) Total Knee Arthroplasty Apparatus and Method of Use

(71) Applicant: Todd Borus, Vancouver, WA (US)

(72) Inventor: Todd Borus, Vancouver, WA (US)

(21) Appl. No.: 13/900,263

(22) Filed: May 22, 2013

Related U.S. Application Data
(60) Provisional application No. 61/650,284, filed on May 22, 2012.

(51) Int. Cl. A61F 2/38; A61B 19/00

(52) U.S. Cl. CPC A61F 2/3859; A61B 19/2203; Y10S 901/41
USPC 606/130; 623/20.35; 901/41

(57) ABSTRACT

A bicruciate-retaining femoral implant component for a knee implant system comprises a first surface comprising two distinct single radii of curvature in a sagittal plane matching a predetermined cylindrical axis of the knee along with a medial surface comprising a first asymmetric geometry substantially congruent with respect to a first native knee geometry and a lateral surface comprising a second asymmetric geometry substantially congruent with respect to the first native knee geometry. In an embodiment, bone resectioning proximate a medial condyle and a posterior condyle for total knee arthroplasty (TKA) comprises using the femoral component after obtaining a pre-operative image of a predetermined region on a knee to be replaced and morphing data collected from the image into a three dimensional model of the knee sufficient to allow modeling software to define a patient specific cylindrical axis. The femoral component is designed as a dual single-radius femoral implant component to conform to a geometry defined about that cylindrical axis.

### RELATION TO OTHER APPLICATIONS

This application claims the benefit of U.S. Provisional Application No. 61/650,284, entitled “Total Knee Arthroplasty Apparatus and Method of Use” and filed on May 22, 2012.

### BACKGROUND

Total knee arthroplasty (TKA) has been documented to be a successful treatment for arthritic conditions of the human knee joint. At a fundamental level, the procedure involves reshaping and resurfacing the medial end of the femur with a metal implant and creating a flat cut on the upper portion of tibia which is capped with a metal implant. A polyethelene liner is then captured into the tibial component, creating an artificial bearing surface between the femoral implant component and polythelene insert and allowing range of motion.

Modern total knee replacement typically relies on at least two of the four major native knee ligaments to provide the construct with inherent stability. These two collateral ligaments, the medial collateral ligament (MCL) and lateral collateral ligament (LCL), are always preserved, but there is great debate in the orthopedic surgery literature as to whether the posterior cruciate ligament (PCL) should be preserved or sacrificed. Implants designed to accommodate sacrificing the posterior cruciate ligament contain a cam and post mechanism on the femoral implant component and poleythelene liner, respectively, to provide the prosthetic knee with restraint against posterior translation of the tibia on the femur.

Although there is debate regarding how best to approach the PCL, modern total knee replacement, by definition, sacrifices the anterior cruciate ligament (ACL). The reason is that when the entire articular surface is removed during TKA, the ACL insertion onto the tibia is concurrently excised.

No currently commercially available total knee replacement designs allow preservation of all four major ligament of the knee: ACL, PCL, MCL, and ACL. Studies have documented that when the ACL is removed with standard TKA, the kinematics of the native knee joint are altered, regardless of implant design. As such, there is potentially great appeal in developing a total knee replacement that preserves both cruciate ligaments, i.e bicruciate-retaining TKA. A successful bi-cruciate sparing TKA would theoretically provide the prosthetic knee joint with greater inherent stability and restore more normal kinematic function.

Current total knee replacement femoral implant components comprise a sagittal plane shape which typically incorporates either a “multi-radius” geometry or a “single-radius” geometry centered around the epicondylar axis of the knee (as opposed to the “cylindrical axis” currently proposed). Designing and placing a single-radius femoral implant component at the cylindrical axis of the knee, at the origin of origin sites of the ACL and PCL and the fixed flexion/extension axis of the knee, would fundamentally promote normal cruciate ligament tension throughout knee range of motion.

Current methods for implanting total knee replacement implants render the practicality of bicruciate-retaining TKA difficult. Most current systems require placement of cutting jigs or guides on the surface of the bone and the surgeon manually activates a saw through cutting slots to perform the bone resections on both the femoral and tibial sides. This technique provides several challenges when attempting to preserve a functional, intact ACL.

For successful execution and performance of a bicruciate-retaining TKR, the monolithic femoral implant component must be positioned to allow the ACL and PCL to function at their native tension throughout knee range of motion. There is a fixed flexion/extension axis of the knee and this is coincident with the “cylindrical axis” of the knee. More specifically, this fixed flexion/extension axis of the knee is best approximated by cylinders fit to the circular posterior femoral condyles. Additionally, the cylindrical axis of the knee passes directly through the origins of the ACL and PCL on a cross-section of cadaveric specimens.

### FIGURES

The various drawings supplied herein are representative of one or more embodiments of the present inventions.

FIG. 1 is a side view in partial perspective of a femoral portion of a human knee.

FIG. 2 is a lateral (side) schematic view of proposed femoral implant component geometry, wherein the solid line represents the medial condylar shape and the dashed line represents the lateral condylar morphology, which has a smaller radius of curvature medially and posteriorly; typically the radius of curvature of the anterior aspect of the component (trochlear groove) has a constant radius medially and laterally.

FIG. 3 is a lateral (side view) of the proposed femoral implant component wherein the medial aspect of femoral implant component geometry incorporates a single radius of curvature encompassing the medial and posterior condyles.

FIG. 4 is a lateral (side view) of the proposed femoral implant component illustrating how the lateral aspect of the femoral implant component geometry also incorporates a single radius of curvature encompassing the medial and posterior condyles (dashed line representing the shape of the lateral condylar geometry)

FIG. 5 illustrates a sagittal (lateral) view of a native femur with superimposed femoral implant component wherein the thick solid black line represents proposed femoral implant component shape, matching the asymmetric geometry of the native femur.

FIG. 6 is a schematic representation of a patient about to have a knee replacement illustrating a robot and jig.

### DESCRIPTION OF EMBODIMENTS

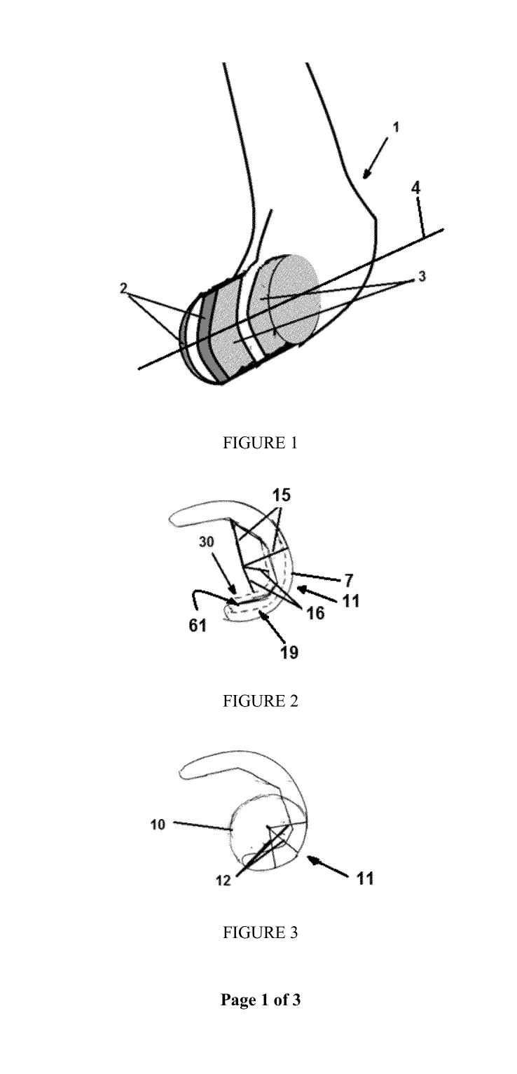

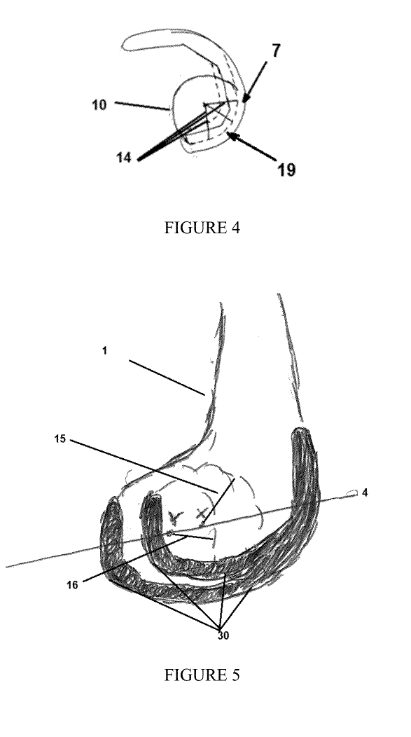

Referring now to FIGS. 1-4, a total knee arthroplasty system comprises bicruciate-retaining femoral implant component 10. At a fundamental level, embodiments of the apparatus described and claimed herein involve an “off the shelf” bicruciate-retaining femoral implant component, e.g. 10, comprising first surface 7 which comprises two distinct single radii of curvature 12 (FIGS. 3) and 14 (FIG. 4) in a sagittal plane matching, and placed accurately on, cylindrical axis 4 of knee 1. As used herein, “sagittal plane” refers to the lateral view of the femur or femoral implant component, as depicted in FIGS. 2,3,4. With respect to FIG. 3, the distance described by radius 12 is a constant radius throughout the flexion range of the component. With respect to FIG. 4, the distance described by radius 14 is a constant radius throughout the flexion range of the component. Though both single radii of curvature, radius 14 is slightly smaller than radius 12, accounting for the asymmetry of normal anatomic femoral morphology. Thus, the propose implant contains dual, but distinct, single radii of curvature on the medial and lateral aspects of the implant.

Femoral implant component 10 further comprises medial surface 11 which comprises first asymmetric geometry substantially congruent with respect to a first native knee geometry, e.g. medial condyle 2; and lateral surface 14 which comprises a second asymmetric geometry substantially congruent with respect to the first native knee geometry, e.g lateral condyle 3. In FIG. 2, medial surface 11 represents an outer surface of a prosthetic medial condyle 2 (FIG. 1); outer later surface 19 represents an outer surface of a prosthetic lateral condyle 3; surface 60 represents an inner surface of a prosthetic lateral condyle 3; and inner surface 61 represents an inner surface of a prosthetic medial condyle 2.

Medial condyle 2 has a slightly larger radius than lateral condyle 3. The asymmetric geometries of medial side 13 and lateral side 14 are configured to comprise a larger radius of curvature medially than laterally. Femoral implant component 10 typically incorporates this asymmetry in its shape and contains a single radius, or circular configuration, posteriorly to allow femoral implant component 10 to match cylindrical axis 4 of knee 1. However, an implant system using femoral implant component 10 may incorporate multiple, different component sizes to accommodate varying patient anatomy.

In embodiments, bone resection around medial condyle 2 and posterior condyle 3 may be accomplished in a manner to substantially exactly match the thickness of an implant using femoral implant component 10 in region 30, thus allowing precise restoration of axis 4.

Referring now to FIG. 5, the smaller lateral condylar implant geometry matches single radius 16 of the native lateral femoral condyle (represented by dashed circle with a center point at origin X of single radius 15) and the larger medial condylar geometry matches single radius 16 of the native medial femoral condyle (represented by dashed line with a center point at origin Y of single radius 16). As such, if the asymmetric, dual single radius component is placed with a center of rotation proximate to X laterally and Y laterally, cylindrical axis 4 will be recreated.

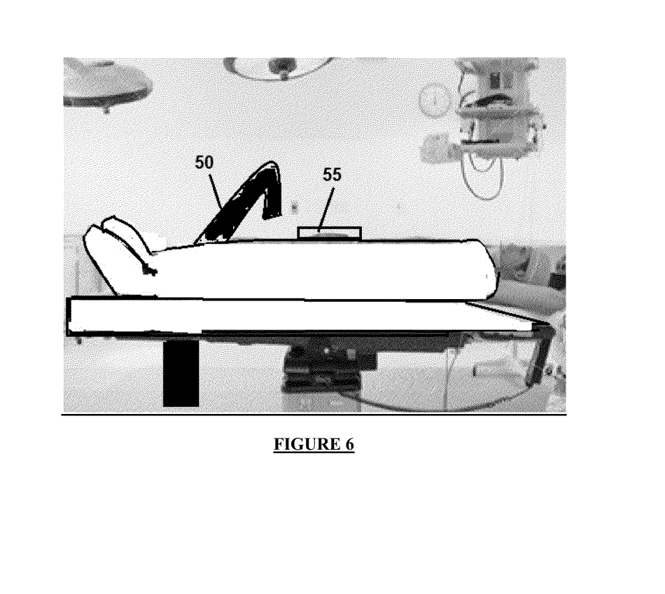

In the operation of exemplary embodiments, standard techniques for implanting TKA femoral implant components impose significant challenges when considering the idea of placing a femoral implant component accurately on a cylindrical axis of the knee. TKA typically involves utilizing various alignment rods and cutting jigs to perform bone cuts and place corresponding components. For such method embodiments, the knee implant system 1 further comprises robot 50 (FIG. 6), used for robotic assistance for precise bone resection, or one or more custom cutting jigs 55 (FIG. 6). Although long term studies have demonstrated success with this method, implanting the femoral implant component accurately along the cylindrical axis of the knee requires the utmost precision for a bicruciate-retaining TKA to succeed.

Recently introduced advances in knee arthroplasty involve robotic-assisted surgery or creating custom cutting jigs to optimize bone resection and alignment angles. Both of these alternative techniques involve obtaining a pre-operative CT or MRI scan to create a virtual, three dimensional model of a patient specific knee. This three dimensional model then guides the planning and execution of the robotically-assisted bone resection or aids engineers in configuring the custom cutting jigs for bone resection to optimally align a standard TKA. This invention further incorporates the information gathered from a pre-operative CT scan or MRI and the ability to morph the collected data into a three dimensional model of the knee to allow modeling software to define a patient specific cylindrical axis. Robotic assistance for precise bone resection or custom cutting jigs, both executed based on the pre-operative CT scan, can then be utilized to accurately perform the procedure and recreate a patient's cylindrical axis with the prosthetic TKA.

Accordingly, bone resectioning proximate medial condyle 2 and posterior condyle 3 for total knee arthroplasty (TKA) may be accomplished by obtaining a pre-operative scan of a predetermined region on a knee to be replaced, where the pre-operative scan may be a CT scan, an MRI scan, or the like, or a combination thereof.

Once obtained, data may be collected [incorporated] from the pre-operative scan and integrated into a three-dimensional model of knee 1 by morphing the collected data sufficient to allow modeling software to define a patient specific cylindrical axis, e.g. cylindrical axis 4.

One or more thicknesses of a femoral implant [substantially exactly] may then be matched to a corresponding area in the predetermined region, thus allowing precise restoration of an axis relative to the femoral implant in the predetermined region. Typically, the thickness of native bone removed matches the exact thickness of the placed implant. Using the matched thicknesses, a dual single-radius femoral implant component may then be designed to conform to a geometry defined about a cylindrical axis of the knee proximate an origin of origin sites of the anterior cruciate ligation (ACL) and the posterior cruciate ligament (PCL), and a fixed flexion/extension axis of the knee.

The designed dual single-radius femoral implant component is then placed at the cylindrical axis of the knee proximate the origin of origin sites of the ACL and PCL and the fixed flexion/extension axis of the knee. The first surface placement may further comprise having dual single radii of curvature in a sagittal plane match accurately on a cylindrical axis of the knee. In certain embodiments, the medial and lateral sides of the joint replacement construct are balanced independently to provide an optimal balance to the cruciate ligaments of the knee.

Once placed, the medial and lateral sides of the prosthetic knee are tensioned to achieve a ligament tension to substantially restore ligament tension in the context of a bicruciate retaining total knew replacement.

When ready, robotic assistance may be used for precise bone resection or a custom cutting jig used to accurately perform the procedure and recreate a patient's cylindrical axis with the prosthetic TKA. The robotic assistance is typically pre-programmed using the pre-operative scan. If used, the custom cutting jig is typically created using the pre-operative scan.

The foregoing disclosure and description of the inventions are illustrative and explanatory. Various changes in the size, shape, and materials, as well as in the details of the illustrative construction and/or an illustrative method may be made without departing from the spirit of the invention.

What is claimed is:

1. A bicruciate-retaining femoral implant component for a knee implant system, comprising:
a) a first surface comprising two distinct single radii of curvature in a sagittal plane matching a predetermined cylindrical axis of the knee;
b) a medial surface comprising a first asymmetric geometry substantially congruent with respect to a first native knee geometry; and
c) a lateral surface comprising a second asymmetric geometry substantially congruent with respect to the first native knee geometry.

2. The knee implant system of claim 1, wherein the asymmetric geometries of the medial and lateral sides are configured to comprise a larger single radius of curvature medially than laterally.

3. A method of bone resectioning proximate a medial condyle and a posterior condyle for total knee arthroplasty (TKA), comprising:
a) obtaining a pre-operative image of a predetermined region on a knee to be replaced;
b) collecting data from the pre-operative image;
c) morphing the collected data into a three dimensional model of the knee sufficient to allow modeling software to define a patient specific cylindrical axis;
d) designing a dual single-radius femoral implant component to conform to a geometry defined about a cylindrical axis of the knee proximate an origin of origin sites of the anterior cruciate ligation (ACL) and the posterior cruciate ligament (PCL), and a fixed flexion/extension axis of the knee, the design comprising matching a thickness of the femoral implant component to a corresponding area in the predetermined region, thus allowing precise restoration of an axis relative to the femoral implant in the predetermined region;
e) placing the dual single-radius femoral implant component at the cylindrical axis of the knee proximate the origin of origin sites of the ACL and PCL and the fixed flexion/extension axis of the knee;
f) tensioning the medial and lateral sides of the prosthetic knee to achieve a ligament tension to substantially restore ligament tension in the context of a bicruciate retaining total knew replacement; and
g) using at least one of robotic assistance for precise bone resection or a custom cutting jig to accurately perform the procedure and recreate a patient's cylindrical axis with the prosthetic TKA.

4. The method of claim 3, wherein the dual single-radius femoral implant component comprises dual but distinct radii.

5. The method of claim 3, further comprising placing the first surface comprising a dual single radii of curvature in a sagittal plane matching accurately on a cylindrical axis of the knee.

6. The method of claim 3, wherein the robotic assistance is programmed using the pre-operative image.

7. The method of claim 3, wherein the custom cutting jig is created using the pre-operative image.

8. The method of claim 3, wherein the pre-operative image is obtained using at least one of a CT scan or an MRI scan.

9. The method of claim 3, wherein the medial and lateral sides of the joint replacement construct are balanced independently to provide an optimal balance to the cruciate ligaments of the knee.

10. The method of claim 3 further comprising applying the design of the femoral implant component to a standard posterior cruciate retaining total knee design.

11. The method of claim 3 wherein matching the thickness of a femoral implant further comprises matching the amount of bone removed from the femoral condyles to the thickness of the femoral implant component replacing the bone.
